(19) United States
(12) Patent Application Publication
(10) Pub. No.: US 2022/0130490 A1
(43) Pub. Date: Apr. 28, 2022

(54) PEPTIDE-BASED VACCINE GENERATION

(71) Applicant: NEC Laboratories America, Inc., Princeton, NJ (US)

(72) Inventors: Renqiang Min, Princeton, NJ (US); Igor Durdanovic, Lawrenceville, NJ (US); Hans Peter Graf, South Amboy, NJ (US)

(21) Appl. No.: 17/510,882

(22) Filed: Oct. 26, 2021

Related U.S. Application Data
(60) Provisional application No. 63/105,926, filed on Oct. 27, 2020.

(51) Int. Cl. G16B 35/00; G16B 40/00; G16B 5/00; G16B 15/00; G06N 3/08; G06N 3/04

(52) U.S. Cl. CPC G16B 35/00; G16B 40/00; G06N 3/04; G16B 15/00; G06N 3/088; G16B 5/00

(57) ABSTRACT

Methods and systems for generating a peptide sequence include transforming an input peptide sequence into disentangled representations, including a structural representation and an attribute representation, using an autoencoder model. One of the disentangled representations is modified. The disentangled representations, including the modified disentangled representation, are transformed to generate a new peptide sequence using the autoencoder model.

### RELATED APPLICATION INFORMATION

This application claims priority to U.S. Provisional Patent Application Ser. No. 63/105,926, filed on Oct. 27, 2020, incorporated herein by reference in its entirety.

### BACKGROUND

### Technical Field

The present invention relates to peptide searching, and, more particularly, to identifying potential new binding peptides with new properties.

### Description of the Related Art

Peptide-MHC (Major Histocompatibility Complex) protein interactions are involved in cell-mediated immunity, regulation of immune responses, and transplant rejection. While computational tools exist to predict a binding interaction score between an MHC protein and a given peptide, tools for generating new binding peptides with new specified properties from existing binding peptides are lacking.

### SUMMARY

A method for generating a peptide sequence includes transforming an input peptide sequence into disentangled representations, including a structural representation and an attribute representation, using an autoencoder model. One of the disentangled representations is modified. The disentangled representations, including the modified disentangled representation, are transformed to generate a new peptide sequence using the autoencoder model.

A method for generating a peptide sequence includes training a Wasserstein neural network model using a set of training peptide sequences by minimizing a mutual information between a structural representation and an attribute representation of the training peptide sequences. An input peptide sequence is transformed into disentangled structural and attribute representations, using an encoder of the Wasserstein autoencoder neural network model. One of the disentangled representations is modified to alter an attribute to improve vaccine efficacy against a predetermined pathogen, including changing coordinates of a vector representation of the disentangled representations within an embedding space. The disentangled representations, including the modified disentangled representation, are transformed to generate a new peptide sequence using a decoder of the Wasserstein autoencoder neural network model.

A system for generating a peptide sequence includes a hardware processor and a memory that stores a computer program product. When executed by the hardware processor, the computer program product causes the hardware processor to transform an input peptide sequence into disentangled representations, including a structural representation and an attribute representation, using an autoencoder model, to modify one of the disentangled representations, and to transform the disentangled representations, including the modified disentangled representation, to generate a new peptide sequence using the autoencoder model.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a diagram illustrating a peptide and a major histocompatibility complex binding, in accordance with an embodiment of the present invention;

FIG. 2 is a block/flow diagram of a method of generating modified peptide sequences with useful attributes, in accordance with an embodiment of the present invention;

FIG. 3 is a block diagram of a Wasserstein autoencoder that generates disentangled representations of an input peptide sequence and modifies the disentangled representations to generate a new peptide sequence, in accordance with an embodiment of the present invention;

FIG. 4 is a block/flow diagram of a method for training a Wasserstein autoencoder to generate disentangled representations of an input peptide sequence, in accordance with an embodiment of the present invention;

FIG. 5 is a block diagram of a computing device that can perform peptide sequence generation, in accordance with an embodiment of the present invention;

FIG. 6 is a block diagram of a peptide sequence generation system that uses a Wasserstein autoencoder to modify attributes of an input peptide sequence and to generate a new peptide sequence, in accordance with an embodiment of the present invention;

FIG. 7 is a diagram illustrating a neural network architecture, in accordance with an embodiment of the present invention; and

FIG. 8 is a diagram illustrating a deep neural network architecture, in accordance with an embodiment of the present invention.

### DETAILED DESCRIPTION OF PREFERRED EMBODIMENTS

Strongly binding peptides can be generated given a set of existing positive binding peptide examples for a major histocompatibility complex (MHC) protein. For example, a regularized Wasserstein autoencoder may be used to generate disentangled representations of a peptide. These disentangled representations may include a first representation of structural information for the peptide, and a second representation for attribute information for the peptide. The disentangled representations may then be altered to change the properties of the peptide, and the autoencoder's decoder may then be used to convert the altered disentangled representations into a new peptide that has the desired attributes.

Prediction of binding peptides for MHC proteins is helpful in vaccine research and design. Once a binding peptide for an MHC protein has been identified, it can be used in the generation of a new peptide vaccine with new properties to target a pathogen, such as a virus. The existing binding peptide may be used as input to the encoder of a learned regularized Wasserstein autoencoder to get disentangled representations, and the decoder of the Wasserstein autoencoder may be used to generate new peptides from the altered representations with new properties. For example, the structural and sequence similarity information of the existing binding peptide may be maintained, but the antigen processing score and T-cell receptor interaction score of the given binding peptide may be increased. The newly generated peptides are similar to the given binding peptide, but may have much higher chances of triggering immune responses corresponding to the targeted T-cell receptors.

Disentangled representation learning maps different aspects of data into distinct and independent low-dimensional latent vector spaces, and can be used to make deep learning models more interpretable. To disentangle the attributes of peptides, two different types of embeddings may be used, including an attribute embedding and a content embedding. The content embedding may encapsulate general structural or sequential constraints of a peptide, while the attribute embedding may represent attributes such as the binding, antigen processing, and T-cell receptor recognition properties of a peptide.

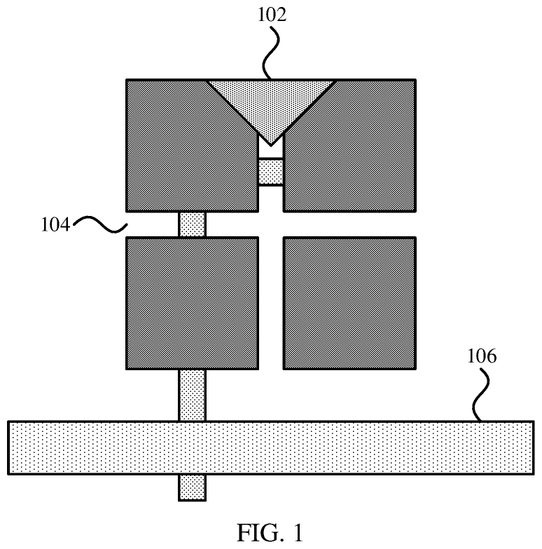

Referring now to FIG. 1, a diagram of a peptide-MHC protein bond is shown. A peptide 102 is shown as bonding with an MHC protein 104, with complementary two-dimensional interfaces of the figure suggesting complementary shapes of these three-dimensional structures. The MHC protein 104 may be attached to a cell surface 106.

An MHC is an area on a DNA strand that codes for cell surface proteins that are used by the immune system. MHC molecules are used by the immune system and contribute to the interactions of white blood cells with other cells. For example, MHC proteins impact organ compatibility when performing transplants and are also important to vaccine creation.

A peptide, meanwhile, may be a portion of a protein. When a pathogen presents peptides that are recognized by a MHC protein, the immune system triggers a response to destroy the pathogen. Thus, by finding peptide structures that bind with MHC proteins, an immune response may be intentionally triggered, without introducing the pathogen itself to a body. In particular, given an existing peptide that binds well with the MHC protein 104, a new peptide 102 may be automatically identified according to desired properties and attributes.

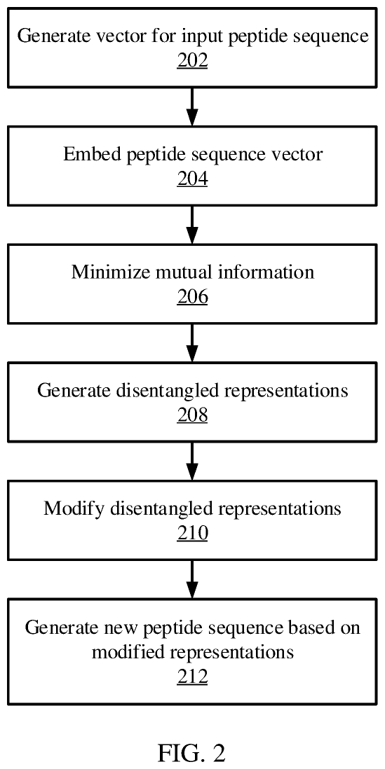

Referring now to FIG. 2, a method for generating new binding peptide sequences is shown. Block 202 accepts an input peptide sequence and generates a vector representation. For example, this vector may be a blocks substitution matrix (BLOSUM) representation of the peptide sequence, which may be implemented as a matrix for sequence alignment of proteins.

Block 204 encodes the input vector using the encoder part of an autoencoder model. As will be described in greater detail below, the encoder part of the autoencoder model translates a vector representation into an embedding in a latent space, and the decoder part translates an embedding in the latent space back into a vector representation. This embedding may include minimization of mutual information between distinct disentangled representations, including a structure representation and an attribute representation, in block 206, thereby generating the disentangled representations in block 208.

Block 210 makes modifications to the disentangled representations. For example, the attributes of the peptide can be altered by moving an attribute representation vector within the latent space. Modifying only the attribute representation, while keeping the structure representation the same, will produce a peptide that is structurally and sequentially similar to the input peptide, but that has the new attributes indicated by the altered attribute representation. As the disentangled representations may be represented by vectors in a latent space, this modification may be performed by changing the coordinates of one or more such vectors.

For example, the attribute representation of the given binding peptide can be replaced with the corresponding attribute representation of another peptide that has high binding affinity, and/or high antigen processing score, and/or high T-cell receptor interaction score. In this way, the newly generated peptide from the altered disentangled representation will have high sequence similarity to the original given binding peptide and the desired attributes.

Block 212 translates the altered disentangled representations back to a peptide sequence vector representation, for example using the decoder part of the autoencoder model. This generates a new peptide sequence that can have high binding affinity, and/or high antigen processing score, and/or high T-cell receptor interaction score.

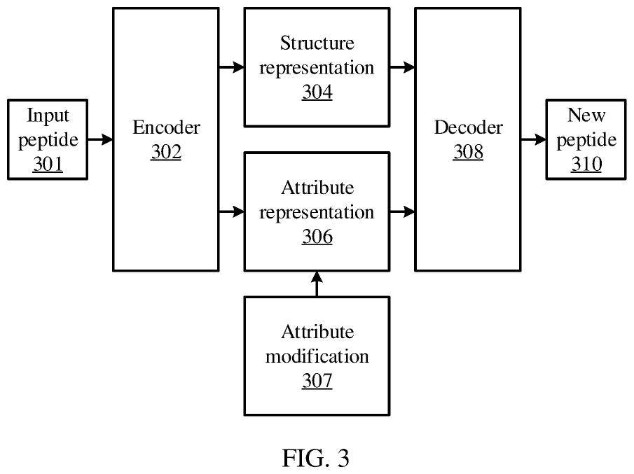

Referring now to FIG. 3, a peptide generation model is shown. An input peptide sequence 301 is provided, for example in the form of a BLOSUM vector. An encoder 302 embeds the input peptide sequence 301 into a latent space, particularly generating disentangled representations that may include a structure or sequence content representation 304 and an attribute representation.

Modifications 307 may be made to the attribute representation 306 in the latent space. When a decoder 308 transforms the structure representation 304 and the modified attribute representation 306/307 into a peptide sequence, the peptide sequence represents a new peptide that includes the new attributes indicated by the modification 307.

The autoencoder that is implemented by the encoder 302 and the decoder 308 may be a Wasserstein autoencoder, implemented as a generative model and trained in an end-to-end fashion. An input peptide x may be encoded into a structure or sequence content embedding s and an attribute embedding a. The attribute embedding a may be classified using a classifier q(y|a) to predict an attribute label y, which may be include experimentally or computationally determined binding affinities. The structure or sequence content embedding s may be used to reconstruct the information of the input peptide x.

A network p(a|s) helps to disentangle the attribute embedding and the structure embedding by minimizing mutual information, while a separate sample-based approximated mutual information term between a and s may also be minimized. The generator p(x|a, s) generates peptides based on the combination of attributes a and structure s, and may represent the decoder 304. Thus, the encoder 302 may be represented by classifier q(a, s|ix), which determines the disentangled representations a and s.

A prior distribution p(a, s)=p(a)p(s) represents the product of two multivariate, isotropic unit-variance Gaussian functions, and may be used to regularize the posterior distribution q(a, s|x) by a Wasserstein distance. The log-likelihood term for the peptide sequence reconstruction may be maximized.

The objective for the encoder may be expressed as:

L_{AE}=W(q(a,s),N(0,1))− [symbol] _{q(a,s|x)}[log p(x|a,s)]

where W(⋅) is the 1-Wasserstein distance metric, which can be approximated by a discriminator as follows:

Eₚ₍ₓ₎(D(z))−Eₚ₍ₓ₎(D(a,s))

where z is sampled from the Gaussian distribution and (a,s) is sampled from the posterior distribution.

A regularization term may be expressed as:

L_{reg}=−log q(y|a)−MI(a;s)

where MI(⋅) is the mutual information and may be expressed as:

MI(a;s)=KL(q(a,s)∥q(a)q(s)=f(q(s,c))−f(q(s))−f(q(c))

where f(⋅)=E_{q(a,s)} log(⋅) and E_{q(a,s)} is the expectation with respect to q(a,s). The term can be approximated using a mini-batch weighted sampling estimator, thus:

$${E}_{q ( a , s )} [ log q ( z ) ] \approx \frac{1}{M} {\sum }_{i = 1}^{M} log {\sum }_{j = 1}^{M} q ( z ( {x}_{i} ) \vert {x}_{j} ) + C$$

where C is a constant and M is the size of the mini-batch.

The final loss function is thus:

L=L_{AE}+λL_{reg}

where λ is a regularization hyper-parameter.

After the regularized autoencoder is trained on a large-scale peptide dataset, which may include attribute and structural/content information, to learn different types of disentangled semantic factors, the disentangled factors (e.g., attribute representation 306, which may include binding/non-binding, high/low antigen processing score, high/low T-cell receptor recognition score, and structural/sequence content representation 304, which may include structural properties) can be replaced for conditional peptide generation. One type of factor may be fixed, such as a high-binding affinity or medium-binding affinity to an MHC protein, and content around the embedding can be sampled from the prior to generate new binding peptides that satisfy different properties.

In the loss function of L_{AE}, a regularization term forces the aggregated latent attribute distribution and the aggregated latent structure/sequence content distribution to follow multivariate unit Gaussian distributions (prior distributions). To sample from the prior distribution of attribute or structure/sequence content vector, a sample can be drawn from a multivariate unit Gaussian distribution while fixing other disentangled latent representations.

The following pseudo-code may be optionally used to further disentangle the attribute representation a from the structural/sequence content representation s besides minimizing the KL-divergence based mutual information above:

```
Input: Data {xⱼ}ⱼ₌₁^{M}, encoder q(a, s|x), approximation network p(a|s)
for (each training iteration) do
  Sample {aₖ, sⱼ}ⱼ₌₁^{M} from q(q, s|x)
  L = 1/M Σⱼ₌₁^{M} log p(aⱼ, sⱼ)
  Update p(a|s) by maximizing L
  for j = 1 to M do
    Sample k′ uniformly from {1, 2, . . . , M}
    \hat{R}ⱼ = log p(aⱼ|sⱼ) − log p(aⱼ|s_{k′})
  end
  $Update q ( a , s \vert x ) by minimizing \frac{1}{M} {\sum }_{j = 1}^{M} {\hat{R}}_{j} + MI ( s \text{;} c )$
End
```

Here M is a mini-batch size. This process may be performed during the training to further disentangle the attribute representation from the sequence content/structural representation.

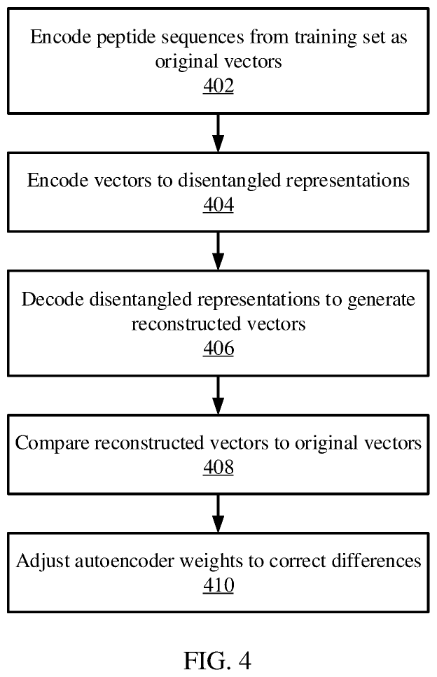

Referring now to FIG. 4, a method of training the autoencoder is shown. Block 402 encodes peptide sequences as original vectors, drawn from a training dataset. Block 404 then uses the encoder 302 to convert the vectors into disentangled representations, including the structural representation 304 and the attribute representation 306.

Block 406 decodes the disentangled representations 304 and 306 to generate reconstructed vectors. Block 408 compares the reconstructed vectors to the original vectors, identifying any differences between the respective pairs. Block 410 updates weights in the encoder 302 and the decoder 308 to correct the differences between the reconstructed vectors and the original vectors. This process may be repeated for any appropriate number of training peptide sequences, for example until a predetermined number of training steps have been performed or until the differences between original vectors and reconstructed vectors drop below a threshold value.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

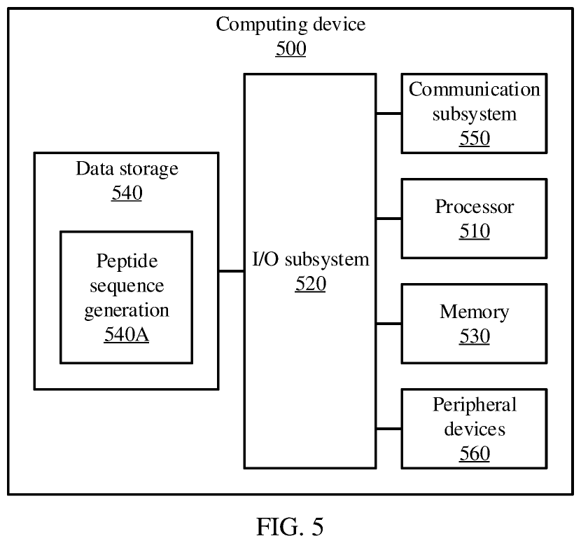

FIG. 5 is a block diagram showing an exemplary computing device 500, in accordance with an embodiment of the present invention. The computing device 500 is configured to identify a top-down parametric representation of an indoor scene and provide navigation through the scene.

The computing device 500 may be embodied as any type of computation or computer device capable of performing the functions described herein, including, without limitation, a computer, a server, a rack based server, a blade server, a workstation, a desktop computer, a laptop computer, a notebook computer, a tablet computer, a mobile computing device, a wearable computing device, a network appliance, a web appliance, a distributed computing system, a processor-based system, and/or a consumer electronic device. Additionally or alternatively, the computing device 500 may be embodied as a one or more compute sleds, memory sleds, or other racks, sleds, computing chassis, or other components of a physically disaggregated computing device.

As shown in FIG. 5, the computing device 500 illustratively includes the processor 510, an input/output subsystem 520, a memory 530, a data storage device 540, and a communication subsystem 550, and/or other components and devices commonly found in a server or similar computing device. The computing device 500 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 530, or portions thereof, may be incorporated in the processor 510 in some embodiments.

The processor 510 may be embodied as any type of processor capable of performing the functions described herein. The processor 510 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 530 may be embodied as any type of volatile or non-volatile memory or data storage capable of performing the functions described herein. In operation, the memory 530 may store various data and software used during operation of the computing device 500, such as operating systems, applications, programs, libraries, and drivers. The memory 530 is communicatively coupled to the processor 510 via the I/O subsystem 520, which may be embodied as circuitry and/or components to facilitate input/output operations with the processor 510, the memory 530, and other components of the computing device 500. For example, the I/O subsystem 520 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 520 may form a portion of a system-on-a-chip (SOC) and be incorporated, along with the processor 510, the memory 530, and other components of the computing device 500, on a single integrated circuit chip.

The data storage device 540 may be embodied as any type of device or devices configured for short-term or long-term storage of data such as, for example, memory devices and circuits, memory cards, hard disk drives, solid state drives, or other data storage devices. The data storage device 540 can store program code 540A for generating peptide sequences. The communication subsystem 550 of the computing device 500 may be embodied as any network interface controller or other communication circuit, device, or collection thereof, capable of enabling communications between the computing device 500 and other remote devices over a network. The communication subsystem 550 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 500 may also include one or more peripheral devices 560. The peripheral devices 560 may include any number of additional input/output devices, interface devices, and/or other peripheral devices. For example, in some embodiments, the peripheral devices 560 may include a display, touch screen, graphics circuitry, keyboard, mouse, speaker system, microphone, network interface, and/or other input/output devices, interface devices, and/or peripheral devices.

Of course, the computing device 500 may also include other elements (not shown), as readily contemplated by one of skill in the art, as well as omit certain elements. For example, various other sensors, input devices, and/or output devices can be included in computing device 500, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 500 are readily contemplated by one of ordinary skill in the art given the teachings of the present invention provided herein.

These and other variations of a hardware processor subsystem are also contemplated in accordance with embodiments of the present invention.

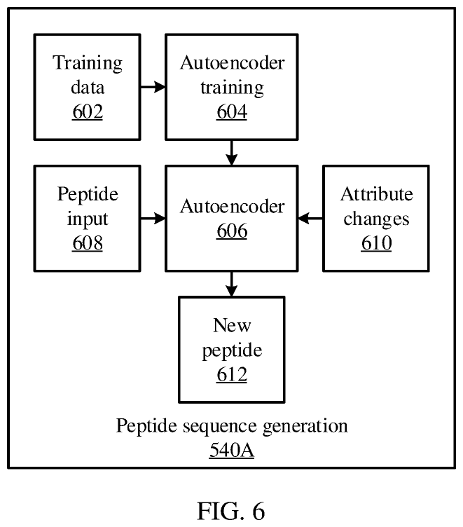

Referring now to FIG. 6, additional detail is shown on the peptide sequence generation 540A. An autoencoder 606 is trained by autoencoder training 604, using a set of training data 602. The training data 602 may be stored, for example in the memory 530 and may be accessed by the peptide sequence generation 540A. As described above, autoencoder training 604 may use the training data 602 as inputs to the autoencoder 606, and may compare the training data 602 to reconstructed outputs from the autoencoder 606, varying parameters of the autoencoder 606 in accordance with the comparison.

During operation, a new peptide input 608 may be applied to the autoencoder 606. Modifications may be made to the disentangled representations, between the operation of the encoder and the decoder of the autoencoder 606. When the decoder of the autoencoder 606 operates on the modified disentangled representations, a new peptide sequence 612 may be generated.

The autoencoder 606 may be implemented in the form of a neural network. In particular, the encoder part and the decoder part may be implemented as respective neural networks of any appropriate depth, with the parameters of each being set to effect the transformation of peptide sequences into embedded representations and the transformation of embedded representations into peptide sequences.

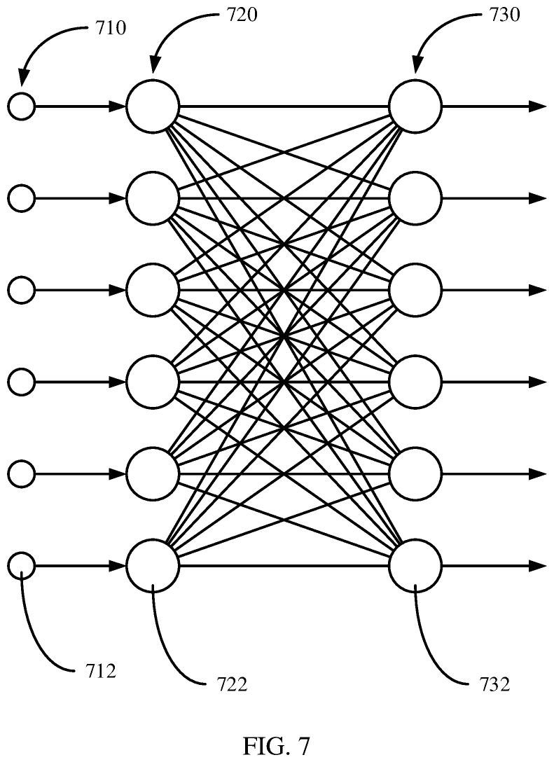

Referring now to FIG. 7, an exemplary neural network architecture is shown. In layered neural networks, nodes are arranged in the form of layers. A simple neural network has an input layer 720 of source nodes 722, a single computation layer 730 having one or more computation nodes 732 that also act as output nodes, where there is a single node 732 for each possible category into which the input example could be classified. An input layer 720 can have a number of source nodes 722 equal to the number of data values 712 in the input data 710. The data values 712 in the input data 710 can be represented as a column vector. Each computational node 730 in the computation layer generates a linear combination of weighted values from the input data 710 fed into input nodes 720, and applies a non-linear activation function that is differentiable to the sum. The simple neural network can perform classification on linearly separable examples (e.g., patterns).

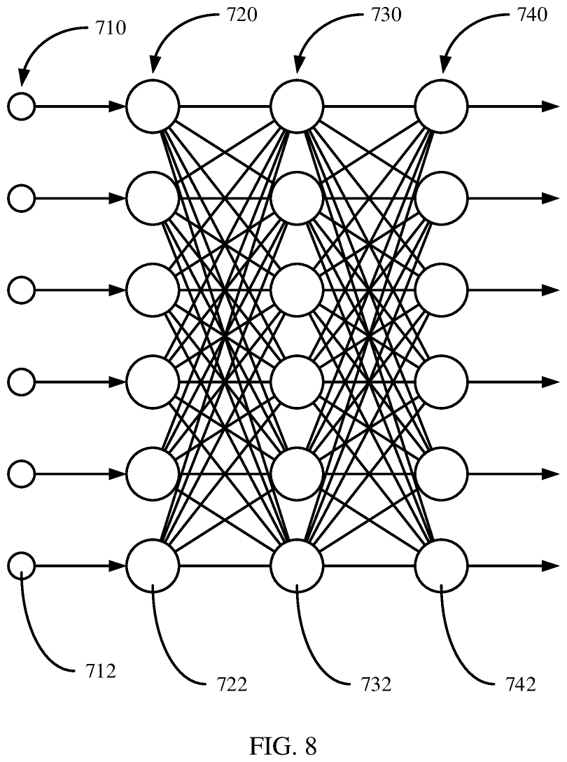

Referring now to FIG. 8, a deep neural network architecture is shown. A deep neural network, also referred to as a multilayer perceptron, has an input layer 720 of source nodes 722, one or more computation layer(s) 730 having one or more computation nodes 732, and an output layer 740, where there is a single output node 742 for each possible category into which the input example could be classified. An input layer 720 can have a number of source nodes 722 equal to the number of data values 712 in the input data 710. The computation nodes 732 in the computation layer(s) 730 can also be referred to as hidden layers because they are between the source nodes 722 and output node(s) 742 and not directly observed. Each node 732, 742 in a computation layer generates a linear combination of weighted values from the values output from the nodes in a previous layer, and applies a non-linear activation function that is differentiable to the sum. The weights applied to the value from each previous node can be denoted, for example, by w₁, w₂, wₙ₋₁ wₙ. The output layer provides the overall response of the network to the inputted data. A deep neural network can be fully connected, where each node in a computational layer is connected to all other nodes in the previous layer. If links between nodes are missing the network is referred to as partially connected.

Training a deep neural network can involve two phases, a forward phase where the weights of each node are fixed and the input propagates through the network, and a backwards phase where an error value is propagated backwards through the network.

The computation nodes 732 in the one or more computation (hidden) layer(s) 730 perform a nonlinear transformation on the input data 712 that generates a feature space. The feature space the classes or categories may be more easily separated than in the original data space.

The neural network architectures of FIGS. 7 and 8 may be used to implement, for example, any of the models shown in FIG. 2. To train a neural network, training data can be divided into a training set and a testing set. The training data includes pairs of an input and a known output. During training, the inputs of the training set are fed into the neural network using feed-forward propagation. After each input, the output of the neural network is compared to the respective known output. Discrepancies between the output of the neural network and the known output that is associated with that particular input are used to generate an error value, which may be backpropagated through the neural network, after which the weight values of the neural network may be updated. This process continues until the pairs in the training set are exhausted.

After the training has been completed, the neural network may be tested against the testing set, to ensure that the training has not resulted in overfitting. If the neural network can generalize to new inputs, beyond those which it was already trained on, then it is ready for use. If the neural network does not accurately reproduce the known outputs of the testing set, then additional training data may be needed, or hyperparameters of the neural network may need to be adjusted.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method of generating a peptide sequence, comprising:
transforming an input peptide sequence into disentangled representations, including a structural representation and an attribute representation, using an autoencoder model;
modifying one of the disentangled representations; and
transforming the disentangled representations, including the modified disentangled representation, to generate a new peptide sequence using the autoencoder model.

2. The computer-implemented method of claim 1, further comprising training a neural network of the autoencoder model using a set of training peptide sequences.

3. The computer-implemented method of claim 2, wherein training the neural network of the autoencoder model includes minimizing a mutual information between the structural representation and the attribute representation.

4. The computer-implemented method of claim 1, wherein modifying the disentangled representations includes modifying a binding affinity.

5. The computer-implemented method of claim 4, wherein the binding affinity is a binding affinity between a peptide and a major histocompatibility complex.

6. The computer-implemented method of claim 1, wherein modifying the disentangled representations includes modifying an antigen processing score.

7. The computer-implemented method of claim 1, wherein modifying the disentangled representations includes modifying a T-cell receptor interaction score.

8. The computer-implemented method of claim 1, wherein modifying the disentangled representations includes altering an attribute to improve vaccine efficacy against a predetermined pathogen.

9. The computer-implemented method of claim 1, wherein modifying the disentangled representations includes changing coordinates of a vector representation of the disentangled representations within an embedding space.

10. The computer-implemented method of claim 1, wherein transforming the input peptide sequence is performed using an encoder of the autoencoder model and transforming the disentangled representations is performed using a decoder of the autoencoder model.

11. A computer-implemented method of generating a peptide sequence, comprising:
training a Wasserstein neural network model using a set of training peptide sequences by minimizing a mutual information between a structural representation and an attribute representation of the training peptide sequences;
transforming an input peptide sequence into disentangled structural and attribute representations, using an encoder of the Wasserstein autoencoder neural network model;
modifying one of the disentangled representations to alter an attribute to improve vaccine efficacy against a predetermined pathogen, including changing coordinates of a vector representation of the disentangled representations within an embedding space; and
transforming the disentangled representations, including the modified disentangled representation, to generate a new peptide sequence using a decoder of the Wasserstein autoencoder neural network model.

12. A system for generating a peptide sequence, comprising:
a hardware processor; and
a memory that stores a computer program product, which, when executed by the hardware processor, causes the hardware processor to:
transform an input peptide sequence into disentangled representations, including a structural representation and an attribute representation, using an autoencoder model;
modify one of the disentangled representations; and
transform the disentangled representations, including the modified disentangled representation, to generate a new peptide sequence using the autoencoder model.

13. The system of claim 12, wherein the computer program product further causes the hardware processor to train a neural network of the autoencoder model using a set of training peptide sequences.

14. The system of claim 13, wherein the computer program product further causes the hardware processor to minimize a mutual information between the structural representation and the attribute representation.

15. The system of claim 12, wherein the computer program product further causes the hardware processor to modify a binding affinity.

16. The system of claim 12, wherein the computer program product further causes the hardware processor to modify an antigen processing score.

17. The system of claim 12, wherein the computer program product further causes the hardware processor to modify a T-cell receptor interaction score.

18. The system of claim 12, wherein the computer program product further causes the hardware processor to alter an attribute to improve vaccine efficacy against a predetermined pathogen.

19. The system of claim 12, wherein the computer program product further causes the hardware processor to change coordinates of a vector representation of the disentangled representations within an embedding space.

20. The system of claim 12, wherein transformation of the input peptide sequence is performed using an encoder of the autoencoder model and transformation of the disentangled representations is performed using a decoder of the autoencoder model.
